(19) United States
(12) Patent Application Publication
(10) Pub. No.: US 2001/0046675 A1
(43) Pub. Date: Nov. 29, 2001

(54) Method and apparatus for producing biochips

(76) Inventor: Takeo Tanaami, Tokyo (JP)

Correspondence Address: MOONRAY KOJIMA, BOX 627, WILLIAMSTOWN, MA, 01267, US

(21) Appl. No.: 09/792,967

(22) Filed: Feb. 26, 2001

(30) Foreign Application Priority Data
May 26, 2000 (JP) 2000/156,231

(51) Int. Cl. C12Q 1/68; C12P 19/34; C12M 1/14; C12M 3/04

(52) U.S. Cl. 435/6; 438/1; 435/91.1; 435/299.1

(57) ABSTRACT

A method and apparatus for quick, easy mass production of biological polymer chips having uniform quality and involving deposition of very small quantities of biological polymers, wherein samples of the biological polymer are deposited on a substrate using a capillary array comprising a plurality of capillaries arranged at the same spacing interval as that of sites on the substrate, and applying an electric field or pneumatic pressure to the array of capillaries containing the biological polymer samples.

### BACKGROUND OF THE INVENTION

1. Field of Invention

This invention relates to a method and apparatus for producing biochips wherein samples of a biological polymer, such as DNA, RNA, protein, sugar chain, and the like, are deposited in an array on a substrate.

2. Description of the Prior Art

Biological polymer chips, such as DNA chips, are generally about 1 to 10 cm² in size and several thousand to several hundred thousand types of DNA segments, or the like are arranged within such an area. Known methods for producing DNA chips, for example, include stamping and depositing a solution of DNA segments, prepared, for example, by polymerase chain reaction (called herein “PCR”) onto a slide glass or silicon substrate using pins on an arrayer. Such stamping and depositing method is described, for example, in U.S. Pat. No. 5,807,522.

The prior art methods and apparatus, however, are plagued by problems. For example, the process using a pin for deposition takes a long time to complete, and the quality of the stamped site, which is also known as a cell or a spot, is not uniform. Moreover, it is difficult with the prior art methods to deposit only a small amount of the biological polymer sample.

### SUMMARY OF THE INVENTION

Accordingly, an object of the invention is to overcome the aforementioned and other problems, deficiencies, and disadvantages of the prior art.

Another object is to provide a method and apparatus for quickly and easily mass producing biological polymer chips having uniform quality and for easily depositing very small amount of biological polymers.

The foregoing and other objects are attained by the invention which encompasses a method for producing biochips by arranging samples of the biological polymer, such as DNA, RNA, protein, or sugar chains, in arrays on a substrate, wherein the samples are deposited onto the substrate using a capillary array comprising a plurality of capillaries arranged at the same spacing interval as that of sites on the substrate, whereby fast, easy mass producing of biological polymer chips having uniform quality is attained.

In one aspect of the invention, the method is implemented by applying a voltage across the capillary array and the substrate so that the biological polymer samples are deposited by effect of the resulting electric field.

In another aspect of the invention, the method is implemented by pressurizing each capillary of the capillary array so that the biological polymer samples are deposited by effect of the pressurization.

Advantageously, with another aspect, it is possible to amplify DNA solutions contained in the capillary array by polymerase chain reaction, thereby saving on labor otherwise required to replenish capillaries with DNA solutions, for example.

With the invention, when using the PCR processing, it is possible to utilize atmospheric temperature change, or heating by laser irradiation.

In a further aspect of the invention, the substrate located is located on either side , i.e. the backside, or the topside, of the capillary array, so that engineering flexibility is provided during construction of the invention.

### BRIEF DESCRIPTION OF THE DRAWING

FIG. 1 is the sole drawing, and is a schematic view depicting an illustrative embodiment of the invention.

### DETAILED DESCRIPTION OF THE PREFERRED EMBODIMENTS

With the invention, a plurality of capillaries, with open ends, are arranged in an array in such a manner that the ends are flush with one another on the same plane. Different types of biological polymer solutions, such as DNA solution, are injected into the capillaries as desired. The array of the capillaries is positioned face to face against a planar substrate, and voltage is applied across the array and substrate. Hence, a biological polymer solution in each capillary is caused to swell out of the bottom end thereof by effect of the electric field, thereby causing droplets of the biological polymer solution, each being in the order of picoliter, to be deposited onto the top surface of the substrate. Using the invention method described, it is possible to easily and quickly produce biological polymer chips, such as DNA chips, having uniform quality.

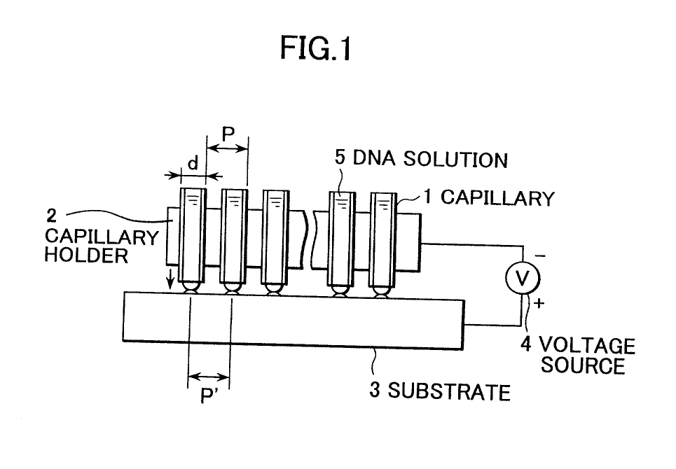

FIG. 1 shows an apparatus for producing the biological polymer chips, such as DNA chips, using the aforementioned method. The invention apparatus comprises a capillary 1, a capillary holder 2, substrate 3, and voltage source 4. A plurality of capillaries 1 are mounted on capillary holder 2 in an array and having the same interval of spacing “P”. A DNA solution 5, for example, is then injected into each capillary 1. The type of capillary used for this purpose has the inside diameter “d” that prevents the solution 5 from spilling out of the capillary bottom end under natural conditions. The plurality of capillaries 1 are mounted vertically on capillary holder 2 so that the bottom ends thereof are flush with one another on the same horizontal plane.

A substrate 3 is a part used to produce a DNA chip, for example, and a top surface thereof is formed so as to be planar. Substrate 3 is arranged so that the top surface thereof is parallel to the bottom ends of capillary 1. Either capillary holder 2, or substrate 3, or both, are disposed to be vertically mobile so that a gap therebetween can be varied, as desired.

A voltage source 4 is used to supply voltage across capillary holder 2 and substrate 3. For example, a positive voltage is supplied to substrate 3 and a negative voltage is supplied to capillary holder 2. When voltage is applied, an electric field acts upon the biological polymer sample, such as DNA solution 5, inside capillary 1, thereby causing the DNA solution to swell below the bottom end of capillary 1 and move toward the top surface of substrate 3.

Operation of the embodiment is as follows. DNA segments 5 (e.g. in solution) are previously injected into the plurality of capillaries 1. Capillaries 1 has suitably thin tubing with inside diameter “d” so that solution 5 will not overflow out of the capillary bottom end. That is the surface tension will exceed the gravitational force. The inside diameter “d” of capillary 1 can be of any value provided it is smaller than spacing “P”.

Capillary holder 2 is moved close to the top surface of substrate 3 so that an appropriate gap is formed therebetween. Then, suitable voltage is supplied from voltage source 4 across capillary holder 2 and substrate 3. Hence, the DNA solution 5 inside capillary 1 is caused to swell below the bottom end of the capillary 1 by effect of the voltage. This causes droplets of the DNA solution 5, which are in the order of picoliter, to be deposited on the top surface of substrate 3. After deposition, application of voltage from source 4 is stopped and capillary holder 2 is moved away from the top surface of substrate 3.

The spacing “P” of the array of capillaries can be adjusted to match the spacing “P′” between the target sites or cells on a DNA chip for example, so that the DNA solution 5 is deposited on all of the sites at the same time, that is concurrently or simultaneously. Accordingly, with this invention method, it is possible to quickly and reliably deposit volumetrically similar droplets of the DNA solution. Also, advantageously, the invention makes possible deposits of very small amounts of DNA solution, such as by use of pipettes.

The foregoing description is only illustrative, and other modifications are equally possible with the invention. For example, each capillary 1 may be pressurized with air or heat or by other means from the side opposite the substrate 3, instead of applying a voltage. Moreover, the positional relationship between substrate 3 and capillary 1 may be reversed vertically. Positioning the substrate 3 above the capillary 1 is advantageous in that the substrate 3 is less likely to become contaminated with dust. Furthermore, PCR may be applied within a capillary, which is advantageous in that then, the only task required is simply to replenish each capillary with a common, amplification purpose solution, thereby saving on labor required otherwise to supply DNA solutions. Also, for heat treatment in PCR, it is possible, with the invention, to cycle the PCR process at higher speeds by atmospheric temperature change or by heating with use of laser irradiation. Known means can be used to irradiate with laser.

Although, DNA was mentioned as a biological polymer, other examples can be used, such as RNA, protein, sugar chain, and the like, for the chips. Furthermore, advantageously, it is possible to concurrently or simultaneously deposit samples of the biological polymer on a plurality of sites or cells on the substrate by applying electric fields or pneumatic pressure to an array of capillaries containing the samples. Accordingly, this invention offers the advantages that biochips are produced quickly, reliably, and that deposited biological polymer samples are substantially volumetrically identical, and very small amounts of the biological polymer samples can be pipetted.

The foregoing description is illustrative of the principles of the invention. Numerous extensions and modifications thereof would be apparent to the worker skilled in the art. All such extensions and modifications are to be considered to be within the spirit and scope of the invention.

What is claimed is:

1. A method of producing biochips by arranging samples of a biological polymer in an array on a substrate, wherein said biological polymer samples are deposited onto said substrate using a capillary array comprising a plurality of capillaries arranged at a same spacing interval as sites on said substrate.

2. The method of claim 1, wherein said biological samples are selected from the group consisting of DNA, RNA, protein and sugar chain, and wherein said samples are deposited by applying voltage across said capillary array and said substrate.

3. The method of claim 1, wherein said biological polymer comprises DNA, and wherein said DNA is contained in said capillary array and is amplified within said capillaries by polymerase chain reaction.

4. The method of claim 1, wherein said biological polymer samples are deposited by pressurizing each capillary of said capillar array.

5. The method of claim 3, wherein temperature processing in said polymerase chain reaction is performed by an atmospheric temperature change or by heating by laser irradiation.

6. An apparatus for producing biochips by arranging samples of a biological polymer in arrays on a substrate, said apparatus comprising:
a capillar holder for supporting a plurality of capillaries arranged at the same spacing interval as that of sites on a biochip;
means for adjusting a gap formed between said capillary holder and said substrate by moving either said capillary holder or said substrate, or both; and
means for transferring said biological polymer samples from said capillaries to said substrate so that said biological polymer samples are deposited thereon.

7. The apparatus of claim 6, further comprising means for amplifying said biological polymer in said capillaries by polymerase chain reaction.

8. The apparatus of claim 6, wherein said substrate is positioned above or below said capillaries.

9. The apparatus of claim 6, wherein said means for transferring comprises a voltage source for applying voltage across said capillary holder and said substrate; and wherein said biological polymer is selected from the group consisting of DNA, RNA, protein, and sugar chain; and wherein said biological polymer is contained in said capillaries and is deposited onto said substrate.

10. The apparatus of claim 9, further comprising means for amplifying said biological polymer in said capillaries by polymerase chain reaction.

11. The apparatus of claim 9, wherein said substrate is positioned above or below said capillaries.

12. The apparatus of claim 6, wherein said means for transferring comprises means for pressurizing said capillaries to cause said biological polymer contained in said capillaries to be deposited onto said substrate.

13. The apparatus of claim 12, further comprising means for amplifying said biological polymer in said capillaries by polymerase chain reaction.

14. The apparatus of claim 12, wherein said substrate is positioned above or below said capillaries.

15. The apparatus of claim 7, wherein temperature processing in said polymerase chain reaction is performed by an atmospheric temperature change or heating by laser irradiation.

16. The apparatus of claim 10, wherein temperature processing in said polymerase chain reaction is performed by atmospheric temperature change or heating by laser irradiation.

17. The apparatus of claim 13, wherein temperature processing in said polymerase chain reaction is performed by an atmospheric temperature change or heating by laser irradiation.

18. The apparatus of claim 7, wherein said substrate is positioned above or below said capillaries.

19. The apparatus of claim 10, wherein said substrate is positioned above or below said capillaries.

20. The apparatus of claim 13, wherein said substrate is positioned above or below said capillaries.
